(19) United States
(12) Patent Application Publication
(10) Pub. No.: US 2004/0241149 A1
(43) Pub. Date: Dec. 2, 2004

(54) Use of unmethylatd cpg

(76) Inventor: Claudio De Simone, Ardea Rm (IT)

Correspondence Address: NIXON & VANDERHYE, PC, 1100 N GLEBE ROAD, 8TH FLOOR, ARLINGTON, VA, 22201-4714, US

(21) Appl. No.: 10/488,606

(22) PCT Filed: Aug. 9, 2002
(86) PCT No.: PCT/IT02/00534

Related U.S. Application Data
(60) Provisional application No. 60/316,953, filed on Sep. 5, 2001.

(51) Int. Cl. A61K 48/00; C12N 1/20

(52) U.S. Cl. 424/93.45; 435/252.3; 435/252.9

(57) ABSTRACT

Lactic acid bacteria containing unmethylated cytosine-guanine (CpG) dinucleotide are used to positively affect the immune response in a subject having or at risk of having an inflammatory response to lipopolysaccharides.

This invention relates to the use of lactic acid bacteria containing unmethylated cytosine-guanine (CpG) dinucleotide to positively affect the immune response in a subject having or at risk of having an inflammatory response to lipopolysaccharides.

### BACKGROUND OF THE INVENTION

Gram-negative infections are a major cause of morbidity and mortality, especially among hospitalized and immunocompromised patients. (Duma, Am. J. of Med., 78 (Suppl. 6A): 154-164, 1985; and Kreger et al., Am. J. Med., 68:344-355, 1980). In most cases, the growth of Gram-negative bacteria can be effectively inhibited by an adequate antibiotic treatment. However, such treatment does not neutralize the pathophysiological effects associated with endotoxin, a potent stimulator of the inflammatory response. Endotoxin is a heat stable bacterial toxin composed of lipopolysacchardes (LPS) released from the outer membrane of Gram-negative bacteria upon lysis (Shenep et al., J. Infect. Dis., 150(3):380-388, 1984). In particular, the penetration of endotoxin into the bloodstream, i.e. endotoxernia, can induce a dramatic systemic inflammatory response.

Morrison et al. (Am. J. Pathol., 93(2):527-617, 1978) have shown that many detrimental in vivo effects of LPS can be attributed to the soluble mediators released by inflammatory cells. In this respect, the role played by monocytes and neutrophils must be emphasized. When in presence with endotoxin in vivo, monocytes and neutrophils release soluble proteins which have microbicidal, proteolytic, opsonic, pyrogenic, complement-activating and tissue-damaging effects. These factors mediate many of the pathophysiological effects of endotoxin. For example, endotoxin-stimulated monocytes can release a particular cytokine called tumor necrosis factor (TNF) which causes fever, shock, and alterations in glucose metabolism.TNF is also a potent stimulator of neutrophils. Other cytokines such as IL-1, IL-6, and IL-8 also mediate many of the pathophysiologic effects of LPS, as well as other pathways involving endothelial cell activation by tissue factor, kininogen, nitric oxide and complement. Various causes can be at the origin of endotoxin-associated disorders, for example, an extra-gastrointestinal exposure to LPS, e.g. administration of LPS-contaminated fluids, inhalation of LPS, or Gram-negative infections. Another cause is when the normal Gram-negative flora breaches the natural cellular barriers (i.e. skin, mucosae, intestinal epithelium) following an injury. Such disorders can also occur for example (a) when there is ischemia of the gastrointestinal tract (e.g, following hemorrhagic shock or during certain surgical procedures), or (b) when there is an increased permeability of the gut or lung to endotoxin or Gram-negative organisms (e.g. in case of systemic or local inflammation).

Gram-negative bacterial infections or endotoxemia can lead to many diseases, such as bacterial meningitis, neonatal sepsis, cystic fibrosis, inflammatory bowel disease and liver cirrhosis, Gram-negative pneumonia, Gram-negative abdominal abscess, hemorrhagic shock and disseminated intravascular coagulation. In particular, leukopenic or neutropenic subjects, including subjects treated with chemotherapy or immunocomprorised subjects (for example with AIDS), are particularly vulnerable to bacterial infection and the subsequent effects of endotoxin.

### DESCRIPTION OF THE INVENTION

The present invention is based on the finding that lactic acid bacteria containing unmethylated CpG affect the immune response in a subject by reducing the inflammatory response to LPS, i.e. by desensitizing the subjects having an excessive Th1 response, or by activating natural killer cells (NK) or redirecting a subject's immune response from a Th2 to a Th1 response by inducing monocytic and other cells to produce Th1 cytokines.

The term “unmethylated CpG” refers to the absence of methylation of the cytosine on the pyrimidine ring.

Lactic acid bacteria containing unmethylated CpG may be selected from Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus cuntatus, Lactobacillus delbrueckii, Lactobacillus fermentum, Lactobacillus gasserii, Lactobacillus jensenii, Lactobacillus leichinanii, Lactobacillus minutus, Lactobacillus plantaruin, Lactobacillus rhamnosus, Lactobacillus rogosae, Lactobacillus salivarius, Bifidobacterium adolescents, Bifidobacterium angulatum, Bifidobacteriumbifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis and Streptococcus thermophilus.

“Cytokine” is a generic term used to indicate a large range of soluble proteins and peptides which act as humoral regulators at nano- to picomolar concentrations. Under normal or pathological conditions, they modulate the functional activities of individual cells and tissues. These proteins also mediate interactions between cells directly and regulate processes taking place in the extracellular environment. Some cytokines, way of example, are TNF-alpha, IL-10, IL-12, and interferon-gamma. In particular, interferon-gamma is a key cytokine for the mediation of LPS-induced inflammation (e.g., Ozmen, L., et al., J. Exp. Med. 180:907-915, 1994). The release of interferon-.gamma is induced by IL-12 derived from macrophage/monocyte/dendritic cells. (e.g., Balanchard, D. K., et al., J. Immunol. 136:963-970, 1986), and IL-10 inhibits interferon-.gamma. via a macrophage-dependent step in which IL-12 production is inhibited (D'Andrea, a., et al., J. Exp. Med. 178:1041-1048, 1993).

Cytokines also play a role in directing the T cell response. Helper (CD4⁺) T cells orchestrate the immune response of mammals through production of soluble factors that act on other immune system cells, including other T cells. Most mature CD4⁺ T helper cells express one of two cytokine profiles: Th1 or Th2. Th1 cells secrete IL-2, IL-3, IFN-gamma, TNF-.beta., GM-CSF and high levels of TNF-alpha. Th2 cells express IL-3, IL-4, IL-5, IL-6, IL-9, IL-10, IL-13, GM-CSF and low levels of TNF-alpha. The Th1 subset promotes delayed-type hypersensitivity, cell-mediated immunity, and immunoglobulin class switching to IgG₂ₐ. The Th2 subset induces humoral immunity by activating B cells, promoting antibody production, and inducing class switching to IgG₁ and IgE.

This invention is an advancement in respect to the actual methods to prepare and administer unmethylated CpG. A number of well-known procedures can be used to synthesize the nucleic acids de novo such as the b-cyanoethyl phosphoramidite method (Beaucage, S. L., and Caruthers, M. H., Tet. Let. 22:1859, 1981); nucleoside H-phosphonate method (Garegg et al., Tet. Let. 27:40514054, 1986; Froehler et al., Nucl. Acid. Res. 14:5399-5407, 1986, Garegg et al., Tet. Let. 27:4055-4058, 1986, Gaffney et al., Tet. Let. 29:2619-2622, 1988). For such purposes, a wide variety of automated oligonucleotide synthesizers are available on the market. Alternatively, CpG dinucleotides can be produced on a large scale in plasmids, (see Sambrook, T., et al., Molecular Cloning: A Laboratory Manual, Cold Spring Harbor laboratory Press, New York, 1989) which after being administered to a subject are degraded into oligonucleotides. Such oligonucleotides can be obtained from existing nucleic acid sequences (e.g., genomic or cDNA) using known techniques, such as those employing restriction enzymes, exonucleases or endonucleases.

For administration in vivo, nucleic acids may be associated with a molecule that results in higher affinity binding to target cell (e.g., B-cell, monocytic cell and natural killer (NK) cell) surfaces and/or increased cellular uptake by target cells to form a “nucleic acid delivery complex.” Nucleic acids can be ionically or covalenfly associated with appropriate molecules using techniques which are well known in the art. A variety of coupling or cross-linking agents can be used, e.g., protein A, carbodiimide, and N-succinimidyl-3-(2-pyridyldithio)propionate (SPDP). Nucleic acids can alternatively be encapsulated in liposomes or virosomes using well-known techniques. This rather complicated approach is justified by the observation that bacterial extracts containing CpG sequences exhibit varying degrees of toxicity, regardless of the route of administration.

Synthetic CpG ODNs mimic bacterial DNA to ‘trick’ the immune system into thinking that an infection has occurred, leading to the initiation of remarkably effective immune defense mechanisms.

This approach has been justified by the observation that crude bacterial extracts have an unacceptable toxicity. In fact, it is well known that mammalian immune system has evolved mechanisms to recognize and respond to “danger” signals arising from bacteria, and that among these danger signals are the unmethylated CpG dinucleotide motifs found in bacteria (Immunotherapy Weekly, March 7, pag. 9, 2001). Therefore bacterial extracts are not considered a suitable way to induce therapeutic responses in humans and animals.

The inventor has surprisingly found that lactic acid bacteria—as live cells or as sonicate—can be an inexpensive and effective source of unmethylated CpG. It should be stressed that this approach is unexpected since the general belief is that bacteria cannot be utilized safely in humans and animals.

To activate the immune response, any of the above listed lactic acid bacterium containing unmethylated CpG can be used either on its own or in combination with another therapeutic modality like a drug or a surgical procedure. In this latter case, lactic acid bacterium containing unmethylated CpG can be administered before, after, and/or simultaneously with the other therapeutic modality.

As a preferred aspect of this invention an sphingomyelinase is administered in addition to the unmethylated CpG. Administration maybe before, after or concurrently with the unmethylated CpG. It has been found in the skin, mucosa, intestinal brush borders and in the bile. It has now been found, surprisingly, that some bacteria possess high levels of sphingomyelinase, and that their ingestion can be beneficial for the host. These bacteria can be ingested live or in the form of extracts, provided that these are enzymatically active, possibly in combination with other bacteria such as lactic acid bacteria, with SM and/or with foods containing SM.

Preferably the sphingomyelinase is of bacterial origin, and the bacteria containing the sphingomyelinase are chosen from among Gram-positive bacteria, Gram-negative bacteria and lactic acid bacteria, or from mixtures thereof.

More especially, the sphingomyelinase is obtained from lactic acid bacteria, and these are chosen from the group comprising Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus jensenii, Lactobacillus leichmannii, Lactobacillus minutus, Lactobacillus plantarum, Lactobacillus rogosae, Lactobacillus salivarius, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis and Streptococcus thermophilus.

The particularly preferred strain amongst these lactic acid bacteria is Lactobacillus brevis CD2, filed on Feb. 6, 1998 under access No. DSM 11,988 in the German Collection of Micro-organisms and Cell Cultures (DSM) in Braunschweig, Germany (“Deutsche Sammlung von Mikroorganismen und Zellkulturen GmbH”) under the Budapest Treaty, or mutants or derivatives thereof.

According to a preferred embodiment of the invention, the lactic acid bacteria are used in the composition as live, lyophilized or sonicated bacteria.

When the compositions of the invention contain sphingomyelinase preferably they contain from 1×10² to 1×10¹³ CFUs of lactic acid bacteria per gram of composition. A particularly preferred composition contains 200×10⁹ Streptococcus thermophilus, 150×10⁹ Bifidobacteria and 4×10⁹ Lactobacillus acidophilus per gram of composition.

In addition to the lactic acid bacteria containing unmethylated CpG and sphingolyelinase the compositions according to the invention can also contain bile acids, in particular ursodeoxycholic acid, pectin, sphingomyelin or its compounds, drugs or foods containing sphingomyelin, argane deirminase, fatty acids, polyunsaturated fatty acids, non fermented sugars, in particular lactulose, cholesterol inhibitors, anti-lipenic agents, ceramidase inhibitors, protease inhibitors, immunomodulators, anti-carcinogenic agents, vitamins, growth factors, surfactants, cereals, fibre, emulsifiers, stabilizers, lipids, antioxidants, preservatives, free-radical neutralizers and/or vaso-protectors.

The composition of the invention can be administered orally as a food supplement or orally or parenterally as a drug or topically as a cream.

The pharmaceutical compositions according to the invention are in general administered topically, intravenously, orally, otically, ophtalmically, intraperitoneously, parenterally or by inhalation or as implants. Rectal use can also be envisaged. Granules, powders, tablets, coated tablets, (micro)capsules, suppositories, syrups, emulsions, suspensions, creams, aerosols, drops or injectable solution in ampule form are all suitable solid or liquid pharmaceutical preparation forms. Also suitable the preparations with protracted release of active compounds, in whose preparation excipients and additives and/or auxiliaries such as disintegrants, binders, coating agents, swelling agents, lubricants, flavorings, sweeteners or solubilizers are customarily used as described above. A variety of drug delivery systems can be applied to these pharmaceutical compositions.

The pharmaceutical compositions are preferably presented in dose units. Solid dose units are tablets, capsules and suppositories. The daily doses recommended for the treatment of a patient will be adjusted individually, taking into account the activity of the compound, mode of administration, nature and severity of the disorder, age and body weight of the patient. Certain specific circumstances may justify the administration of a higher or lower daily dose. The daily dose can be administered in different ways, e.g. as a single administration (either one individual dose unit or several smaller dose units) and also by multiple administration (dose unit subdivided and administered at specific intervals).

Suitable pharmaceutical presentations include aqueous and nonaqueous isotonic sterile solutions for nasal, optic or optic administration by instillation in the nose, ear or eye as well as by inhalation either as a solution or a powder. Such solutions may contain anti-oxidants, buffers, bacteriostats and solutes which render the formulation isotonic with the body fluids of the intended recipient. In addition the aqueous and nonaqueous sterile suspensions may include suspending agents and thickening agents. Suitable pharmaceutical compositions are creams, ointments and other preparations for topical applications.

### DETAILED DESCRIPTION OF THE INVENTION

The present invention is based on the finding that lactic acid bacteria containing unmethylated cytosine-guanine (CpG) dinucleotide positively affect the immune response in a subject having or at risk of having an inflammatory response to LPS. Many diseases can be attributed to an inflammatory reponse to LPS such as bacterial meningitis, neonatal sepsis, cystic fibrosis, inflammatory bowel disease and liver diseases, Gram-negative pneumonia, Gram-negative abdominal abscess, hemorrhagic shock and disseminated intravascular coagulation.

Subjects who are leukopenic or neutropenic, including subjects treated with chemotherapy or immunocompromised subjects (for example with AIDS), are particularly vulnerable to bacterial infection and the subsequent effects of endotoxin and are therefore a specific target of this invention.

Lactic acid bacteria containing unmethylated CpG have been shown to activate natural killer cells (NK) or redirect a subject's immune response from a Th2 to a Th1 response by inducing monocytic and other cells to produce Th1 cytokines. Therefore, in another embodiment of this invention, lactic acid bacteria containing unmethylated cytosine-guanine (CpG) dinucleotides, can be used to treat disorders having increased Th2 responses.

Some such disorders (Th2) are atopic disorders in genetically susceptible individuals, stagional rhinitis, asthma, contact dermatitis, atopic dermatitis, urticaria, Omenn's syndrome, idiopathic pulmonary fibrosis, progressive systemic sclerosis, systemic lupus erythematosus, chronic graft-versus host disease, respiratory syncytial virus bronchiolitis, pemphigus vulgaris, AIDS, ulcerative colitis, oral diseases, stomatitis, gengivitis, aphtous ulcers, vulvovaginitis, mucous membrane eczema, epididymitis, prostatitis, bronchiectasis, angioneurotic edema, insect bites, burns.

In another embodiment of this invention, an effective amount of lactic acid bacteria containing unmethylated CpG is administered prophylactically to desensitize the subjects at risk for an immunological disorder characterized by an excessive Th1 immune response Some such disorders (Th1) are organ-specific autoinmmune disorders, insulin-dependent diabetes mellitus, Multiple Sclerosis and other encephalomyelitis, Glomerulomephritis, Reumatoid Arthritis and other arthritides, Autoimmune Uveitis and other inflammatory diseases of the eye, conjunctivitis, keratitis, Encephalomyelitis, Crohn's disease, Helicobacter pylori-induced peptic ulcer, acute kidney allograft rejection, hepatopaties, hepatities, hepatic steatosis, steatohepatitis, unexplained recurrent abortions, otitis and other inflammatory diseases of the ear.

The present invention applies also to methods and products for including a synergistic immune response using in combination a lactic acid bacteria and a cytokine and/or a vaccine. In one aspect the invention includes a method for stimulating an immune response in a subject. The method includes the steps of administering an effective amount of an immunopotentiating cytokine and/or vaccine and an effective amount of lactic acid bacteria containing unmethylated CpG in order to induce a synergistic antigen specific immune response in a subject exposed to a specific antigen.

In another embodiment, lactic acid bacteria containing unmethylated CpG can be used as vaccine adjuvants and in the field of cancer immunotherapy. The lactic acid bacteria can be used as per se, in lyophilized form, or as sonicates or extracts.

Isolation of neutral, acidic and shingomyelinase in lactic acid bacteria is as follows: 10 mg of lyophilized Streptococcus thermophilus bacteria were suspended in 500 μl of a buffer containing 50 mM Tris-HCl, pH 7.4, 10 mM MgCl₂, 2 mM EDTA, 5 mM DTT, 0.1 mM Na₃VO₄, 0.1 mM Na₂MoO₄, 30 mM p-nitrophenyl phosphate, 10 mM β-glycerophosphate, 750 mM ATP, 1 μl PMSF, 10 μM leupeptin, 10 μl pepstatin (from Sigma Chemical Co.) and 0.2% Triton X-100 (to assay the activity of neutral SMase) or 500 μl of 0.2% Triton X-100 (to assay the activity of acidic SMase). To assay the alkaline SMase, the bacteria and the (homogenized) intestinal biopsy material were suspended in a 0.25 M sucrose buffer containing 5 mM MgCl₂, 0.15 M KCl, 50 mM KH₂PO₄, 1 mM PMSF and 1 mM benzamidine (pH 7.4). The samples prepared in this way were then subjected to lysis by sonication (for 30 min during which, 10-sec “on” periods alternated with 10-sec “off” periods), using a Vibracell sonicator (Sonic and Materials Inc., Danbury, Conn.). The sonicated samples wore then centrifuged for 30 min at 14,000 rpm at 4° C., the supernatant was removed and the protein concentration was determined with a kit made by Bio-Rad Laboratories. (Richmond, Calif.).

To determine the neutral SMase, 100 μg of the sample were incubated for 2 hours at 37° C. in a buffer (final volume: 50 μl) containing 50 mM Tris-HCl, 1 mM MgCl₂, pH 7.4, and 2.25 Hl of [N-methyl-¹⁴C]-sphingomyelin (SM) (0.2 μCi/ml, specific activity: 56.6 mCi/mmol, Amersham).

To determine the activity of the acidic sphingomyelinase, 100 μg of the bacterial lysate were incubated for 2 hours at 37° C. in a buffer (final volume: 50 μl) containing 250 mM sodium acetate, 1 mM EDTA, pH 5.0, and 2.25 μl of [N-methyl-¹⁴C]-SM.

To assay the alkaline SMase, the samples were added to 375 μl of Tris-EDTA buffer (pH 9) to a final volume of 0.4 ml, containing 50 mM Tris, 0.15 M NaCl, 2 mM EDTA and a mixture of 3 mM bile salts with a TC:TDC:GC:GCDC molar ratio of 3:2:1.8:1. This mixture of bile salts had been found to possess the highest stimulatory effect on alkaline SMase. The addition of EDTA to the buffer served to inhibit the activity of neutral SMase, which is Mg⁺⁺-dependent with an optimum pH of 7.5. The ¹⁴C-SM was dissolved in ethanol, dried under nitrogen and suspended in the assay buffer, containing a mixture of 3% Triton X-100 and 3 mM bile salts.

The reaction was terminated by the addition of 2 ml of a 2:1 mixture of chloroform and methanol. The phospholipids were extracted and analysed on TLC plates, while the hydrolysis of the SM was quantified by autoradiography and liquid scintillation counting. The SMase present in the sonicated bacteria and in the intestinal biopsy material was expressed as pmol of SM hydrolysed per hour per milligram of protein.

Activity levels of sphingomyelinase in sonicated Streptococcus thermophilus were studied. No activity due to acidic SMase was found, but appreciable levels of both neutral and alkaline SMase were observed in the bacterial samples tested under the experimental conditions used (various pH values and with and without MgCl2).

An assay of the activity of SMase in the samples of Streptococcus thermophilus, under the experimental conditions used for the determination of intestinal SMase, showed that the bacterial enzyme was not affected by the presence or absence of bile salts. Furthermore, when the bacterial SMase activity and the intestinal SMase activity were tested simultaneously, the hydrolysis of SM increased additively. Similar results (not shown) were obtained with the Lactobacillus brevis CD2 strain, filed on Feb. 6, 1998 under access No. DSM 11,988 in the German Collection of Microorganisms and Cell Cultures in Braunschweig, Germany (“Deutsche Sammlung von Mikroorganismen und Zellkulturen GmbH”) under the Budapest Treaty, or mutants or derivatives thereof.

### EXAMPLE

In the present experiment, we evaluated the tissue level of pro- and anti-inflammatory cytokines, nitric oxide synthase and matrix metalloproteinases in control and inflamed pouches before and after lactic acid bacteria treatment of patients with acute pouchitis. The obtained results demonstrated that lactic acid bacteria treatment of patients with pouchitis is able to induce, in the mucosal pouch, a significant increase in the expression of the anti-inflammatory cytokine IL-10, with respect to inflamed untreated patients. In addition, it was observed that inflamed pouch tissue shows increased levels of the inducible nitric oxide synthase (iNOS) and matrix metalloproteinases (MMP) activity. Both iNOS and MMP activity were reduced following lactic acid bacteria treatment.

Patients

A total of 12 patients with ileal pouch anal anastomosis (IPAA) for ulcerative colitis were examined in an unblinded fashion. These patients, of which 7 with acute inflammation of the pouch and 5 with no pouch inflammation, represent a subset of patients enrolled in a clinical trial previously described and selected in an unbiased way (P. Gionchetti et al. Oral bacteriotherapy as maintenance treatment in patients with chronic pouchitis: a double blind, placebo-controlled trial. Gastroenterology 2000; 119(2): 305-9. 7 patients were treated with a highly concentrated probiotic mixture containing 300 billion viable lyophilized bacteria per gram of 4 strains of lactobacilli (Lactobacillus plautarum, Lactobacillus casei, Lactobacillus acidophilus, Lactobacillus delbrueckii subspecies bulgaricius), 3 strains of bifidobacteria (Bifidobacterium infantis, Bifidobactenium longum, Bifidobacterium breve) and 1 strain of Streptococcus salivarius subspecies therinophilus, given orally twice daily (2×3 gram per day) for 9 months—see the strains proposed in this preparation contain unmethylated CpG. No patient had a relapse in this period while, as previously described, 100% of patients receiving placebo had a relapse during the 9 months follow-up period.

Tissue Sample Preparation and Cytokine Measurement

During endoscopic control 4 tissue specimens were taken (the average weight of one biopsy was 5 mg) and immediately washed in 2 ml phosphate-buffered saline containing protease inhibitors (1 μg/ml of leupeptin, 1 g/ml pepstatin-A, 1 μl/ml aprotinin). The biopsies were subsequently homogenised using a Ultra Turrax homogenizer, centrifuged at 1800 g for 10 min and supernatants frozen to −80° C. until assay. Proteins were assayed using a modified Lowry method (Petersen G L. A simplification of the protein assay method of Lowry et al which is more generally applicable. Anal Biochem 1987;83:346-356). All cytokines in supernatants were measured by ELISA using commercial kits obtained from Endogen, Inc. (Woburn, Mass., USA). Results are expressed in pg per mg protein.

Substrate Gel Electrophoresis (Zymogram)

SDS-PAGE zymograms containing either 0.1% gelatin or 0.1% casein were performed as previously described using approximately 2 μg and 10 μg, of sample protein from pouch tissue to reveal the gelanolytic and caseinolytic activity, respectively (Thorgeirsson U P, Mackay A R. Characterization of metastatic tumor cell. In: Gallegner C T, Rees R C, Reynolds C W, eds. Tumor immunology: a pratical approach. Oxford: University Press, 1992:82-90). Following electrophoresis, gels were rinsed in 50 mM Tris-HCl (pH 7.4) containing 2% Triton X-100 followed by 50 mM Tris-HCl (pH 7.4) and incubated overnight for gelatinases or 72 h for caseinases, in a buffer containing 50 mM Tris-HCl (pH 7.4), 0.2 M NaCl, 5 mM CaCl₂, 1% Triton-X100 at 37° C. In some experiments, to test the effect of the different components of the preparation on MMP activity, 0.5 mg/lrn of bacterial extracts were included in the incubation buffer. Enzyme activity was detected following staining with 0.1% Coomassie blue in a mixture of acetic acid:methanol:water (1:3:6), destained in the same mixture without dye and dried. Sample collagenase activity was compared with that of partially purified gelatinases (MMP-2 and MMP-9) obtained from the supernatants of HT 1080 human fibrosarcoma cell line stimulated with TPA (10⁻⁷M) for 48 h (Okada Y, Gonoji Y, Naka K, et al. Matrix metalloproteinase 9 (92 kDa gelatinase/type IV collagenase) from HT 1080 human fibrosarcoma cells. J Biol Chem 1992;267:21712-21719). Sample caseinase activity was similarly compared with the previously characterized caseinase activity present in the supernatant of human breast carcinoma cell line, MDA-MB-2331 (MDA) cell line conditioned for 48 h (Mackay A R, Ballin M, Pelina M D, et al. Effect of phorbol ester and cytokines on matrix metalloproteinase and tissue inhibitor of metalloproteinase expression in tumor and normal cell lines. Invasion Metastasis 1992;12:168-184 and Festuccia C, Bologna M, Vicentini C, et al. Increased matrix metalloproteinase-9 secretion in short term tissue cultures of prostatic tumor cells. Int J Cancer 1996;69:386-393). This cell line expresses a single 53 kDa band of EDTA-inhibitable caseinolvtic activity in zymograms (MMP-3).

Dot Blot Analysis

Tissue sample proteins (2.5 and 5 μg of each sample) diluted in TBS were loaded on nitrocellulose transfer membrane (Protran®, Schleicher & Schuell Inc., Keene, USA) using a dot blot apparatus (EuroClone. Pero, Milan, Italy) under vacuum, washed once with 0.5 ml of TBS (20 mM Tris, 13-I muM NaCl, pH 7.6) and once with TBS-T (TBS, 0.3% Tween20). Non specific binding sites were blocked with 10% non-fat dry milk in TBS-T for 16 h at 4° C. Membrane was then washed 3 times for 10 min at room temperature with TBS-T and incubated for 1 h at room temperature with a mouse monoclonal anti-human MMP-9 (Caibiochem-Niovabiochem Co., Darrnstadt, Germany), diluted 1:1000 in 5% non-fat dry milk in TBS-T. At the end of the incubation, membranes were washed and incubated for 1 h at room temperature with a goat anti-mouse-horseradish peroxidase conjugated IgG (Santa Cruz Biotechnology Inc., Santa Cruz, Calif., USA) diluted 1:1000 in 5% non-fat drv milk in TBS-T. After the incubation, membranes were washed and immunoreactivity assessed by chemiluminescence reaction using the ECL Western blotting detection system (Pierce, Ill., Rockford, USA).

Determination of Nitric Oxide Synthase (NOS) Activity.

NOS activity was determined by measuring the conversion of [³H]-L-arginine to [³H]-L-citrulline. Pouch tissue samples were homogenized by sonication in ice-cold homogenization buffer (25 mM Tris-HCl, pH 7.4, 1 μM EDTA, 1 mM EGTA) containing protease inhibitors (0.2 mM PMSF, 10 μg/ml aprotinin, 10 p gm/ml pepstatin A, 10 μg/ml leupeptin). The homogenates were centrifuged at 4° C. for 20 min at 10,978 g. The supernatants were passed through an AG50WX-8 Dowex resin (Bio-Rad Laboratories, Melville, N.Y.) to remove endogenous arginine. Sample proteins were measured through the Pierce Micro BCA assay kit (Pierce, Rockford, Ill., USA), with bovine serum albunlin as standard. Pouch tissue extracts (15 μg) were incubated at 37° C. for 30 min in 50 mM Tris-HCl, pH 7.4, containing 6 μM (6R)-5,6,7,8-tetrahydro-biopterin (H₄B), 2 μM flavin adenine dinucleotide (FAD), 2 μM flavin mononucleotide (FMN), 1 mM nicotinamide adenine dinucleotide phosphate (NADPH), 1 mM CaCl₂, 1 mM MgCl₂, 1 mM L-citrulline (to prevent the catabolism of [³H]-L-citrulline), 60 mM L-valine, 60 mM L-ornithine, 60 mM L-lysine (to inhibit arginase activity) (Kaysen G A, Strecker H J. Purification and properties of arginase of rat kidney. Biochem J 1973;133:779-788) and 10 μCi/nl of [³H]-L-arginine (specific activity 66 Ci/miol, Amersham). The reaction was stopped by the addition of 0.4 ml of stop buffer (50 mM Hepes, pH 5.5, 5 mM EDTA). The samples were spotted onto TLC plates and developed in chloroform/methanol/ammonium hydroxide/water (1:4:2:1, by vol.) as a solvent system. After drying, the radiolabeled spots were visualised after spraying EN³HANCER by autoradiography. ³H-arginine and ³H-citrulline, identified by co-chromatbgraphy with unlabeled standards, which were revealed by ninhydrin spray, were scraped, and quantified by liquid scintillation. Sample enzyme activity was also tested in the presence of 20 mM formarnidine (arginine deiminase inhibitor) (Weickmann J L, Himmel M E, Smith D W, et al. Arginine deiminase: demonstration of two activity sites and possible half-of-the-sites reactivity. Biochem Biophys Res Cormmun 1978;83:107-113) or 10 μM aminoguanidine (specific iNOS inhibitor) (Misko T P, Moore W M, Kasten T P, et al. Selective inhibition of the inducible nitric oxide synthase by aminoguanidine. Eur J Pharmacol 1993;233:119-125) or 500 μM L-NAME (constitutive and inducible NOS inhibitor) (Kubes P, Suzuki M, Granger D N. Nitric oxide: an endogenous modulator of leukocyte adhesion. Proc Natl Acad Sci USA 1991;88:4651-4655 and Reif D W, McCreedy S A. N-nitro-L-arginine and N-monomethyl-L-arginine exhibit a different pattern of inactivation toward the three nitric oxide synthase. Arch Biochem Biophys 1995;320:170-176) to discriminate arginine to citrulline conversion due to arginine deiminase and/or inducible or constitutive nitric oxide synthase activity, respectively.

Statistical Analysis

Tissue level of cytokines and NOS activity were expressed as the median±SD. Statistical significance of differences between control and inflamed pouch tissue level of cytokines and NOS activity was analyzed by the nonparametric Wilcoxon 2-tailed test while cytokines and NOS activity tissue level differences following patient treatments were analyzed by the Mann-Whitney U test. Values from zymogram analysis were expressed as the mean±SE of three experiments and were statistically compared using the Student's t-test. Results were considered to be significantly different if p values were lower than 0.05.

### Results

Clinical Observations

During the following 9 months therapy with the lactic acid bacteria all 7 patients remained in remission as judged by clinical, endoscopic and histological criteria.

Cytokine Evaluation

A statistically significant increase (p<0.01) in the levels of the pro-inflammatory cytokines TNF-α (18.2±14.7 pg/mg prot) was observed in inflamed pouches compared to uninflamed pouch values (TNF-(×2.1±0.6 pg/mg prot). Tissue levels of IFN-γ (117±105 pg/mg prot) and IL-1α (19.0±11.9 pg/mg prot) were also elevated in pouchitis relative to control pouch values (IFN-γ 43±35 pg/mg prot; IL-1α 2.5±6.6 pg/mg prot) but did not reach the statistical significance. On the contrary, no major differences in the levels of the anti-inflammatory cytokines IL4 (1.7±1.5 vs 2.5±1.5 pg/mg prot of control pouches) and IL-10 (8.54±6.0 vs 7.0±2.7 pg/mg prot of control pouches) could be observed. Following 9 months treatment with the lactic acid bacteria we could observe a statistical decrease (p<0.05) of TNF-((3.7±4.9 pg/mg prot), IL-1α (4.0±3.7 pg/mg prot) and IFN-γ (27.0±41 pg/mg prot). Finally, while we did not notice any modification of IL-4 level (0.9±3.4 pg/mg prot), a significant increase (p<0.05) in the tissue level of IL-10 (44.7±25 pg/mg prot) was observed following lactic acid bacteria treatment.

Iniducible Nitric Oxide Synthase (iNOS) Activity

We therefore monitored NOS activity levels in control and inflamed pouch tissue, as well as following probiotic treatment. The results of these experiments show a significant increase (p<0.05) of the iNOS activity, expressed as citrulline formation from arginine substrate, in inflamed pouches (1.9±0.6 pmol/mg protein/min) compared to control pouches (0.6±0.2 pmol/mg protein/min). This decrease was pronounced, and statistically significant (p<0.05), following treatment with lactic acid bacteria (0.5±0.2 pmol/mg protein/mnin).

Matrix Metalloproteinase (MMP) Activity

Expression and activation of MMPs play an important role in the evolution of the inflammatory processes, as well as in the pathogenesis of inflammatory disease. Both macrophages and T cells have been shown to produce several MMPs, including MMP-1, MMP-2, MMP-3, MMP-9 and MMP-12, and their expression appears to be modulated by cytokines (Goetzl E J, Banda M J, Leppert D. Matrix metalloproteinases in immunity. J Immunol 1996;156:1-4-Xia M, Leppert D, Hauser S L, et al. Stimulus specificity of matrix metalloproteinase dependence of human T cell migration through a model basement membrane. J Immunol 1996;156:160-167 and Shapiro S D, Kobayashi D K, Pentland A P, et al. Induction of macrophage metalloproteinases by extracellular matrix. Evidence for enzyme- and substrate-specific responses involving prostaglandin-dependent mechanisms. J Biol Chem 1993;268:8170-8175). We here investigated the level of gelatinases and caseinases in control pouches, inflamed pouches and following lactic acid bacteria treatment. Control uninflamed pouches did not show appreciable gelatinase activity in contrast to a strong gelatinase activity, mainly MMP-9 and to minor extent MMP-2, present in the inflamed pouches. In all 7 patients very little or no gelatinase activity could be observed following probiotic treatment. The reduction in MMP-9 activity was mirrored by the reduction of MMP-9 protein, as demonstrated by dot blot experiments, using a specific anti-MMP-9 antibody.

1. A method of preventing or treating an inflammatory condition comprising administering to a subject in need of same a lactic acid bacteria containing unmethylated cytosine-guanine dinucleotide to modulate the Th2 and/or Th1 response of a human's or animal's immune system.

2. The method according to claim 1, wherein lactic acid bacteria are administered in combination with a cytokine and/or a vaccine to boost the subject's immune response.

3. A method of treating or preventing organ-specific autoimmune disorders, insulin-dependent diabetes mellitus, Multiple Sclerosis and other encephalomyelitis, glomerulomephritis, reumatoid Arthritis and other arthritides, autoimmune uveitis and other inflammatory diseases of the eye, conjunctivitis, keratitis, Encephalomyelitis, Crohn's disease, Helicobacter pylori-induced peptic ulcer, acute kidney allograft rejection, unexplained recurrent abortions, otitis and other inflammatory diseases of the ear, atopic disorders in genetically susceptible individuals, stagional rhinitis, asthma, contact dermatitis, atopic dermatitis, urticaria, Omenn's syndrome, idiopathic pulmonary fibrosis, progressive systemic sclerosis, systemic lupus erythematosus, chronic graft-versus host disease, respiratory syncytial virus bronchiolitis, pemphigus vulgaris, AIDS, ulcerative colitis, oral diseases, stomatitis, gengivitis, aphtous ulcers, vulvovaginitis, mucous membrane eczema, epididymitis, prostatitis, bronchiectasis, angioneurotic edema, insect bites and burns,
said method comprising administering to a subject in need of same a lactic acid bacterial containing an unmethylated cytosine-guanine dinucleotide.

4. A method of reducing metalloproteinases in humans and animals comprising administering to a subject in need of same an unmethylated cytosine-guanine dinucleotide from a lactic acid bacteria.

5. A method of reducing apoptosis, caspases or cytokines IL-1β, TNF-α or γ IFN in humans or animals comprising administering to a subject in need of same an unmethylated cytosine-guanine dinucleotide from a lactic acid bacteria.

6. A method according to any one of claims 1, 3, 4 and 5 wherein the lactic acid bacteria is selected from the group consisting of Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus gasserii, Lactobacillus jensenii, Lactobacillus leichmanii, Lactobacillus minutus, Lactobacillus plantarum, Lactobacillus rhamnosus, Lactobacillus rogosae, Lactobacillus salivarius, Bifidobacterium adolescents, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis, Streptococcus thermophilus and mixtures thereof.

7. The method of any one of claims 1, 3, 4 and 5 wherein the lactic acid bacteria are administered in combination with a cytokine and/or a vaccine to boost the host's immune response.

8. The method of any one of claims 1, 3, 4 and 5 wherein the bacteria is administered orally, topically, rectally, otically, ophthalmically or by inhalation.

9. A method of treating inflammatory lesions, fistulas and/or healing of wounds comprising administering to a subject in need of same lactic acid bacteria containing unmethylated cytosine-guanine dinucleotide optionally in combination with a growth factor, a cytokine or an antibody.

10. A method of administering a lactic acid bacteria selected from the group consisting of Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus gasserii, Lactobacillus jensenii, Lactobacillus leichmanii, Lactobacillus minutus, Lactobacillus plantarum, Lactobacillus rhamnosus, Lactobacillus rogosae, Lactobacillus salivarius, Bifidobacterium adolescents, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis, Streptococcus thermophilus or mixtures thereof containing unmethylated cytosine-guanine dinucleotide to treat and/or prevent organ-specific autoimmune disorders, insulin-dependent diabetes mellitus, multiple sclerosis and other encephalomyelitis, glomerulomephritis, reumatoid arthritis and other arthritides, autoimmune uveitis and other inflammatory diseases of the eye, conjunctivitis, keratitis, encephalomyelitis, Crohn's disease, Helicobacter pylori-induced peptic ulcer, acute kidney allograft rejection, unexplained recurrent abortions, otitis and other inflammatory diseases of the ear, atopic disorders in genetically susceptible individuals, stagional rhinitis, asthma, contact dermatitis, atopic dermatitis, urticaria, Omenn's syndrome, idiopathic pulmonary fibrosis, progressive systemic sclerosis, systemic lupus erythematosus, chronic graft-versus host disease, respiratory syncytial virus bronchiolitis, pemphigus vulgaris, AIDS, ulcerative colitis, oral diseases, stomatitis, gengivitis, aphtous ulcers, vulvovaginitis, mucous membrane eczema, epididymitis, prostatitis, bronchiectasis, angioneurotic edema, insect bites, burns.

11. The method of claim 10 wherein the lactic acid bacteria are administered in lyophilized form, as a sonicate and/or an extract.

12. A composition for the prevention or treatment of inflammatory conditions or to reduce metalloproteinases in humans or animals, said composition comprising lactic acid bacteria containing unmethylated cytosine-guanine dinucleotide, said bacteria selected from the group consisting of Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus gasserii, Lactobacillus jensenii, Lactobacillus leichmanii, Lactobacillus minutus, Lactobacillus plantarum, Lactobacillus rhamnosus, Lactobacillus rogosae, Lactobacillus salivarius, Bifidobacterium adolescents, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis, Streptococcus thermophilus and mixtures thereof.

13. A composition for nasal, rectal, opthalmic, bronchial or rectal administration for the prevention or treatment of inflammatory conditions or to reduce metalloproteinases in humans or animals, said composition comprising lactic acid bacteria containing unmethylated cytosine-guanine dinucleotide, said bacteria selected from the group consisting of Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus gasserii, Lactobacillus jensenii, Lactobacillus leichmanii, Lactobacillus minutus, Lactobacillus plantarum, Lactobacillus rhamnosus, Lactobacillus rogosae, Lactobacillus salivarius, Bifidobacterium adolescents, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis, Streptococcus thermophilus and mixtures thereof.

14. A method of preventing or treating an inflammatory condition comprising administering to a subject in need of same a lactic acid bacteria containing unmethylated cytosine-guanine dinucleotide in combination with a lactic acid bacteria containing alkaline sphingomyelinase to modulate the Th2 and/or Th1 response of a human's or animal's immune system.

15. The method according to claim 14, wherein lactic acid bacteria are administered in combination with a lactic acid bacteria containing alkaline sphingomyelinase and cytokine and/or a vaccine to boost the subject's immune response.

16. A method of treating or preventing organ-specific autoimmune disorders, insulin-dependent diabetes mellitus, Multiple Sclerosis and other encephalomyelitis, Glomerulomephritis, Reumatoid Arthritis and other arthritides, Autoimmune Uveitis and other inflammatory diseases of the eye, conjunctivitis, keratitis, Encephalomyelitis, Crohn's disease, Helicobacter pylori-induced peptic ulcer, acute kidney allograft rejection, unexplained recurrent abortions, otitis and other inflammatory diseases of the ear, atopic disorders in genetically susceptible individuals, stagional rhinitis, asthma, contact dermatitis, atopic dermatitis, urticaria, Omenn's syndrome, idiopathic pulmonary fibrosis, progressive systemic sclerosis, systemic lupus erythematosus, chronic graft-versus host disease, respiratory syncytial virus bronchiolitis, pemphigus vulgaris, AIDS, ulcerative colitis, oral diseases, stomatitis, gengivitis, aphtous ulcers, vulvovaginitis, mucous membrane eczema, epididymitis, prostatitis, bronchiectasis, angioneurotic edema, insect bites and burns, said method comprising administering to a subject in need of same a lactic acid bacterial containing an unmethylated cytosine-guanine dinucleotide in combination with a lactic acid bacteria containing alkaline sphingomyelinase.

17. A method of reducing metalloproteinases in humans and animals comprising administering to a subject in need of same an unmethylated cytosine-guanine dinucleotide from a lactic acid bacteria in combination with a lactic acid bacteria containing alkaline sphingomyelinase.

18. A method of reducing apoptosis, caspases or cytokines IL-1β, TNF-α or γ IFN in humans or animals comprising administering to a subject in need of same an unmethylated cytosine-guanine dinucleotide from a lactic acid bacteria in combination with a lactic acid bacteria containing alkaline sphingomyelinase.

19. A method according to any one of claims 14, 16, 17 and 18 wherein the unmethylated cytosine-guanine dinucleotide containing lactic acid bacteria is selected from the group consisting of Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus gasserii, Lactobacillus jensenii, Lactobacillus leichmanii, Lactobacillus minutus, Lactobacillus plantarum, Lactobacillus rhamnosus, Lactobacillus rogosae, Lactobacillus salivarius, Bifidobacterium adolescents, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis, Streptococcus thermophilus and mixtures thereof.

20. A composition for the prevention or treatment of inflammatory conditions or to reduce metalloproteinases in humans or animals, said composition comprising lactic acid bacteria containing unmethylated cytosine-guanine dinucleotide in combination with a lactic acid bacteria containing alkaline sphingomyelinase, said bacteria selected from the group consisting of Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus gasserii, Lactobacillus jensenii, Lactobacillus leichmanii, Lactobacillus minutus, Lactobacillus plantarum, Lactobacillus rhamnosus, Lactobacillus rogosae, Lactobacillus salivarius, Bifidobacterium adolescents, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis, Streptococcus thermophilus and mixtures thereof.

21. A composition for nasal, rectal, opthalmic, bronchial or rectal administration for the prevention or treatment of inflammatory conditions or to reduce metalloproteinases in humans or animals, said composition comprising lactic acid bacteria containing unmethylated cytosine-guanine dinucleotide in combination with a lactic acid bacteria containing alkaline sphingomyelinase, said bacteria selected from the group consisting of Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus gasserii, Lactobacillus jensenii, Lactobacillus leichmanii, Lactobacillus minutus, Lactobacillus plantarum, Lactobacillus rhamnosus, Lactobacillus rogosae, Lactobacillus salivarius, Bifidobacterium adolescents, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis, Streptococcus thermophilus and mixtures thereof.

22. The composition according to claim 20 or 21., wherein the alkaline sphingomyelinase is of bacterial origin, and the bacteria containing the alkaline sphingomyelinase are chosen from amongst Gram-positive bacteria, Gram-negative bacteria and lactic acid bacteria, or from mixtures thereof.

23. The composition according to claim 22, wherein the alkaline sphingomyelinase is obtained from lactic acid bacteria are selected from the group comprising Lactobacillus acidophilus, Lactobacillus brevis, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus fermentum, Lactobacillus jensenii, Lactobacillus leichmanii, Lactobacillus minutus, Lactobacillus plantarum, Lactobacillus rogosae, Lactobacillus salivarius, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudocatenulatum, Bifidobacterium pseudolongum, Streptococcus lactis, Streptococcus raffinolactis and Streptococcus thermophilus.

24. The composition according to claim 23, wherein the Lactobacillus brevis CD2 strain is accession No. DSM 11,988 or mutants or derivatives thereof.

25. The composition according to claim 22, wherein the lactic acid bacteria are used in the composition as live, lyophilized or sonicated bacteria.

26. The composition according to claim 21 or 22, containing from 1×10² to 1×10¹³ CFUs of lactic acid bacteria per gram of composition.

27. The composition according to claim 26, wherein the composition contains 200×10⁹ Streptococcus thermophilus, 150×10⁹ Bifidobacteria and 4×10 Lactobacillus acidophilus per gram of composition.

28. The composition according to claim 20 or 21 which also contains bile acids, in particular ursodeoxycholic acid, pectin, sphingomyelin or its compounds, drugs or foods containing sphingomyelin, arginine deiminase, fatty acids, polyunsaturated fatty acids, non fermented sugars, in particular lactulose, cholesterol inhibitors, ceramidase inhibitors, protease inhibitors, immunomodulators, anti-carcinogenic agents, vitamins, growth factors, surfactants, cereals, fiber, emulsifiers, stabilizers, lipids, antioxidants, preservatives, free-radical neutralizers and/or vaso-protectors.
